SEQUENCE LISTING (59 sequences)

SEQ ID NO: 1 - protein, 62 aa, Bitis arietans arietans
  bitoran D protein isolated from venom of Bitis arietans arietans
SKKRPDFCYLPADDGPCRAFIPSFYYNSTSNECNTFIYGGCYGNANKFESMDECRKTCVASA

SEQ ID NO: 2 - protein, 63 aa, Bitis arietans arietans
  bitoran V protein isolated from venom of Bitis arietans arietans
CRQNRPDFCYLPAVEGPCRAYIRSFFYNSTSNECEKFFYGGCYGNANKFETRDECRKTCVASA

SEQ ID NO: 3 - protein, 67 aa, Bitis arietans
  variant of bitoran D protein
SKKRPDFCYLPADDGPCRAYIPSFYYNSTSNECNTFIYGGCYGNANKFESMDECRKTCVASATRRPT

SEQ ID NO: 4 - DNA, 436 bp, Bitis arietans
  cDNA of mRNA coding proprotein of variant bitoran D protein
atgtcttctggaggtctgcttctcctgctgggactcctcatcctctggacagagcagacccccgtgtccagcaagaaacgtccagacttttgttatctccctgctgatgatggaccatgtagggcctatatcccttccttctactacaactcgacatcaaacgaatgtaatacatttatttatggtggatgctatgggaatgccaacaaatttgagagcatggatgaatgtcgcaagacgtgtgttgcatccgcaacgaggagacccacctgaattgggtctaattcgccaacttgtctgagaggcttttkttctgtcctggattgttccagagaccaccccccacaaacccatcctggcttcatcccttctgttctgcaataaagctttgcctctcgctgcaraaaaaaaaaaaaaaaaaaaaaaaaaaaaaaaa

SEQ ID NO: 5 - protein, 90 aa, Bitis arietans
  deduced amino acid sequence for proprotein of variant bitoran D protein
MSSGGLLLLLGLLILWTEQTPVSSKKRPDFCYLPADDGPCRAYIPSFYYNSTSNECNTFIYGGCYGNANKFESMDECRKTCVASATRRPT

SEQ ID NO: 6 - DNA, 30 bp, Artificial Sequence
  forward PCR primer for cloning cDNA coding Kunitz type inhibitor proteins from snake venom
atgtcttctggaggtcttcttctcctgctg

SEQ ID NO: 7 - protein, 62 aa, Artificial Sequence
  Modified protein of bitoran D protein
SKKRPDFCYLPAXXGPCRAXIXSFXYNSTSNECXXFXYGGCYGNANKFEXXDECRKTCVASA

SEQ ID NO: 8 - protein, 63 aa, Artificial Sequence
  Modified protein of bitoran V protein
CRQNRPDFCYLPAXXGPCRAXIXSFXYNSTSNECXXFXYGGCYGNANKFEXXDECRKTCVASA

SEQ ID NO: 9 - protein, 5 aa, Unknown
  partial amino acid sequence of bovine pancreas trypsin inhibitor
CKARI

SEQ ID NO: 10 - protein, 5 aa, Unknown
  partial amino acid sequence of TFPI Kunitz II
CRGYI

SEQ ID NO: 11 - protein, 5 aa, Unknown
  partial amino acid sequence of bitoran D
CRAFI

SEQ ID NO: 12 - protein, 5 aa, Unknown
  partial amino acid sequence of bitoran V
CRAYI

SEQ ID NO: 13 - protein, 59 aa, Artificial Sequence
  partial amino acid sequence of modified bitoran V protein
RPDFCYLPAXXGPCRAXIXSFXYNSTSNECXXFXYGGCYGNANKFEXXDECRKTCVASA

SEQ ID NO: 14 - protein, 19 aa, Unknown
  partial amino acid sequence of bitoran D
GPCRAFIPSXXXXXXXXXC

SEQ ID NO: 15 - protein, 20 aa, Unknown
  partial amino acid sequence of bitoran V
GPCRAYIRSXXXXXXXXXXC

SEQ ID NO: 16 - protein, 19 aa, Artificial Sequence
  partial amino acid sequence of modified bitoran V protein
GPCRAXIXXXXXNSTSNEC

SEQ ID NO: 17 - protein, 15 aa, Bitis arietans
  bitoran protein leader sequence
MSSGGLLLLLGLLTL

SEQ ID NO: 18 - protein, 90 aa, Bitis gabonica
  Kunitz type protein with trypsin inhibitor activity
MSSGGLLLLLGLLTLWAELTPVSGKKRPDFCYLPADTGPCMANFPRFYYDSASKKCKKFTYGGCHGNANNFETREECRKKCFASAARRPT

SEQ ID NO: 19 - protein, 90 aa, Bitis gabonica
  Kunitz type protein with trypsin inhibitor activity
MSSGGLLLLLGLLTLWAELTPVSGKNRPEFCNLPADTGPCKAYEPRFYYDSVSKECQKFTYGGCKGNSNNFESMDECRKTCVASATRRPT

SEQ ID NO: 20 - protein, 65 aa, Vipera ammodytes ammodytes
  Kunitz type protein with trypsin inhibitor activity
RDRPKFCYLPADPGRCLAYMPRFYYNPASNKCEKFIYGGCRGNANNFKTWDECRHTCVASGIQPR

SEQ ID NO: 21 - protein, 93 aa, Vipera ammodytes
  Kunitz type protein with trypsin inhibitor activity
MSSGGLLLLLGLLTLWAELTPVSTRDRPKFCYLPADPGRCLAYMPSFYYDSASNKCKKFIYGGCRGNANNFKTWDECRHTCVASGIQPRIASN

SEQ ID NO: 22 - protein, 62 aa, Oxyuranus scutellatus scutellatus
  Kunitz type protein with trypsin inhibitor activity
KDRPKFCHLPPKPGPCRAAIPRFYYNPHSKQCEKFIYGGCHGNANKFKTPDECNYTCLGVSL

SEQ ID NO: 23 - protein, 62 aa, Eristocophis macmahonii
  Kunitz type protein with trypsin inhibitor activity
FCYLPDDPGVCKAHIPRFYYNPASNKCKNFIYGGCGGNANNFETRAECRHTCVASGKGGPRP

SEQ ID NO: 24 - protein, 57 aa, Hemachatus haemachatus
  Kunitz type protein with trypsin inhibitor activity
RPDFCELPAETGLCKAYIRSFHYNLAAQQCLQFIYGGCGGNANRFKTIDECRRTCVG

SEQ ID NO: 25 - protein, 59 aa, Daboia russellii siamensis
  Kunitz type protein with trypsin inhibitor activity
HDRPTFCNLAPESGRCRGHLRRIYYNLESNKCKVFFYGGCGGNANNFETRDECRETCGG

SEQ ID NO: 26 - protein, 57 aa, Naja nivea
  Kunitz type protein with trypsin inhibitor activity
RPRFCELPAETGLCKARIRSFHYNRAAQQCLEFIYGGCGGNANRFKTIDECHRTCVG

SEQ ID NO: 27 - protein, 83 aa, Tropidechis carinatus
  Kunitz type protein with trypsin inhibitor activity
MSSGGLLLLLGLLTLWEGLTPVSSKDRPDFCELPDDRGPCRGIFHAFYYNPDQRQCLEFIYGGCYGNANNFKTIDECERTCAA

SEQ ID NO: 28 - protein, 83 aa, Notechis scutatus scutatus
  Kunitz type protein with trypsin inhibitor activity
MSSGGLLLLLGLLTLWAELTPVSSKDHPEFCELPADSGPCRGILHAFYYHPVHRTCLEFIYGGCYGNANNFKTIDECKRTCAA

SEQ ID NO: 29 - protein, 83 aa, Pseudonaja textilis textilis
  Kunitz type protein with trypsin inhibitor activity
MSSGGLLLLLGLLTLWEVLTPVSSKDRPDFCELPADTGPCRVRFPSFYYNPDEKKCLEFIYGGCEGNANNFITKEECESTCAA

SEQ ID NO: 30 - protein, 83 aa, Oxyuranus microlepidotus
  Kunitz type protein with trypsin inhibitor activity
MSSGGLLLLLGLLTLWEVLTPVSSKDRPDFCELPADTGPCRVGFPSFYYNPDEKKCLEFIYGGCEGNANNFITKEECESTCAA

SEQ ID NO: 31 - protein, 83 aa, Pseudechis australis
  Kunitz type protein with trypsin inhibitor activity
MSSGGLLLLLGLLTLWEELTPVSSKDRPDFCELPADTGPCRVGFPSFYYNPDEKKCLEFIYGGCQGNANNFITKEECESTCAA

SEQ ID NO: 32 - protein, 83 aa, Oxyuranus scutellatus
  Kunitz type protein with trypsin inhibitor activity
MSSGGLLLLLGLLTLWEVLTPVSSKDRPDFCELPADTGPCRVGFPSFYYNPDEKKCLEFIYGGCEGSANNFITKEECESTCAA

SEQ ID NO: 33 - protein, 58 aa, Ophiophagus Hannah
  Kunitz type protein with trypsin inhibitor activity
GRPKFCELPPEPGLCNARKTFFYYSLHSHACQKFIYGGCGGNANKFKTIDECHRTCVG

SEQ ID NO: 34 - protein, 59 aa, Dendroaspis polylepis polylepis
  Kunitz type protein with trypsin inhibitor activity
LQHRTFCKLPAEPGPCKASIPAFYYNWAAKKCQLFHYGGCKGNANRFSTIEKCRHACVG

SEQ ID NO: 35 - protein, 83 aa, Pseudonaja textilis textilis
  Kunitz type protein with trypsin inhibitor activity
MSSGGLLLLLGLLTLWEVLTPVSSKDRPELCELPPDTGPCRVRFPSFYYNPDEQKCLEFIYGGCEGNANNFITKEECESTCAA

SEQ ID NO: 36 - protein, 83 aa, Bungarus candidus
  Kunitz type protein with trypsin inhibitor activity
MSSGGLLLLLGLLTLCAELTPVSSKDRPKFCNVPPEPGRCNANVRAFYYNPRLRKCIEFTYGGCGGNANNFKSRGECKRTCAE

SEQ ID NO: 37 - protein, 57 aa, Dendroaspis angusticeps
  Kunitz type protein with trypsin inhibitor activity
AAKYCKLPVRYGPCKKKIPSFYYKWKAKQCLPFDYSGCGGNANRFKTIEECRRTCVG

SEQ ID NO: 38 - protein, 50 aa, Bitis arietans arietans
  partial amino acid sequence of bitoran D
SKKRPDFCYLPADDGPCRAFIPSFYYNSTSNECNTFIYGGCYGNANKFES

SEQ ID NO: 39 - protein, 51 aa, Bitis arietans arietans
  partial amino acid sequence of bitoran V
CRQNRPDFCYLPAVEGPCRAYIRSFFYNSTSNECEKFFYGGCYGNANKFET

SEQ ID NO: 40 - protein, 46 aa, Vipera ammodytes ammodytes
  partial amino acid sequence of Kunitz type protein with trypsin inhibitor activity
PKFCYLPADPGRCKAHIPRFYYDSASNKCNKFIYGGCPGNANNFKT

SEQ ID NO: 41 - protein, 47 aa, Vipera ammodytes
  partial amino acid sequence of Kunitz type protein with chymotrypsin inhibitor activity
RPKFCYLPADPGRCLAYMPRFYYNPASNKCEKFIYGGCRGNANNFKT

SEQ ID NO: 42 - protein, 50 aa, Pseudonuja textilis textilis
  partial amino acid sequence of Kunitz type protein with plasmin inhibitor activity
SKDRPDFCELPADTGPCRVRFPSFYYNPDEKKCLEFIYGGCEGNANNFIT

SEQ ID NO: 43 - protein, 49 aa, Daboia russellii siamensis
  partial amino acid sequence of Kunitz type protein with trypsin inhibitor activity
HDRPTFCNLAPESGRCRGHLRRIYYNLESNKCKVFFYGGCGGNANNFER

SEQ ID NO: 44 - protein, 47 aa, Hemachatus haemachatus
  partial amino acid sequence of Kunitz type protein with trypsin inhibitor activity
RPDFCELPAETGLCKAYIRSFHYNLAAQQCLQFIYGGCGGNANRFKT

SEQ ID NO: 45 - protein, 47 aa, Naja naja naja
  partial amino acid sequence of Kunitz type protein with trypsin inhibitor activity
RPGFCELPAAKGLCKAHKPAFYYNKDSHRCQKFIYGGCGGNANRFTI

SEQ ID NO: 46 - protein, 48 aa, Ophiagus Hannah
  partial amino acid sequence of Kunitz type protein with chymotrypsin inhibitor activity
GRPKFCELPPEPGLCNARKTFFYYSLHSHACQKFIYGGCGGNANKFTI

SEQ ID NO: 47 - protein, 49 aa, Dendoroaspis polylepis
  partial amino acid sequence of Kunitz type protein with trypsin inhibitor activity
LQHRTFCKLPAEPGPCKASIPAFYYNWAAKKCQLFHYGGCKGNANRFST

SEQ ID NO: 48 - protein, 49 aa, Bungarus fasciatus
  partial amino acid sequence of Kunitz type protein with chymotrypsin inhibitor activity
KNRPTFCNLLPETGRCNALIPAFYYNSHLHKCQKFNYGGCGGNANNFKT

SEQ ID NO: 49 - protein, 47 aa, Dendroaspis angusticeps
  partial amino acid sequence of Kunitz type protein with trypsin inhibitor activity
HRTFCKLPAEPGPCKASIPAFYYNWAAKKCQLFHYGGCKGNANRFST

SEQ ID NO: 50 - protein, 44 aa, Eristocophis macmahonii
  partial amino acid sequence of Kunitz type protein with trypsin inhibitor activity
FCYLPDDPGVCKAHIPRFYYNPASNKCKNFIYGGCGGNANNFET

SEQ ID NO: 51 - protein, 47 aa, Naja nivea
  partial amino acid sequence of Kunitz type protein with trypsin inhibitor activity
RPRFCELPAETGLCKARIRSFHYNRAAQQCLEFIYGGCGGNANRFKT

SEQ ID NO: 52 - protein, 50 aa, Oryctolagus cuniculus
  partial amino acid sequence of Kunitz type protein with trypsin inhibitor activity
QKPTHSFCAMKVDDGPCRAYIKRFFFNILTHQCEEFIYGGCEGNENRFES

SEQ ID NO: 53 - protein, 50 aa, Homo sapiens
  partial amino acid sequence of Kunitz I protein
LKLMHSFCAFKADDGPCKAIMKRFFFNIFTRQCEEFIYGGCEGNQNRFES

SEQ ID NO: 54 - protein, 50 aa, Homo sapiens
  partial amino acid sequence of Kunitz II protein
QQEKPDFCFLEEDPGICRGYITRYFYNNQTKQCERFKYGGCLGNMNNFET

SEQ ID NO: 55 - protein, 50 aa, Homo sapiens
  partial amino acid sequence of Kunitz III protein
EFHGPSWCLTPADRGLCRANENRFYYNSVIGKCRPFKYSGCGGNENNFTS

SEQ ID NO: 56 - protein, 47 aa, Xenopus laevis
  partial amino acid sequence of Kunitz type protein with trypsin inhibitor activity
VREVCSEQAETGPCRAMISRWYYDVTESKCAQFIYGGCGGNRNNFES

SEQ ID NO: 57 - protein, 44 aa, Homo sapiens
  partial amino acid sequence of Kunitz type protein with trypsin inhibitor activity
ACNLPIVQGPCRAFIKLWAFDAAQGKCIQFHYGGCKGNGNKFYS

SEQ ID NO: 58 - protein, 44 aa, Mus musculus
  partial amino acid sequence of Kunitz type protein with trypsin inhibitor activity
ACNLPIVRGPCRAFIQLWAFDAVKGKCVLFPYGGCQGNGNKFYS

SEQ ID NO: 59 - protein, 44 aa, Equus caballus
  partial amino acid sequence of Kunitz type protein with trypsin inhibitor activity
ACNLPIVQGPCRAFIRLWAFDAAQGKCVLFTYGGCRGNGNQFYS

(19) United States
(12) Patent Application Publication
(10) Pub. No.: US 2009/0036647 A1
(43) Pub. Date: Feb. 5, 2009

(54) BITORAN WHICH IS TRYPSIN-INHIBITOR-LIKE PROTEIN DERIVED FROM BITIS ARIETANS VENOM, AND USE THEREOF AS BLOOD COAGULATION FACTOR Xa INHIBITOR

(75) Inventor: Takashi Morita, Saitama (JP)

Correspondence Address: SUGHRUE MION, PLLC, 2100 PENNSYLVANIA AVENUE, N.W., SUITE 800, WASHINGTON, DC, 20037, US

(73) Assignees: Takashi Morita, Tokorozawa-shi (JP); NEC SOFT, LTD., Tokyo (JP)

(21) Appl. No.: 11/916,621

(22) PCT Filed: Jun. 6, 2005
(86) PCT No.: PCT/JP2005/010335

§ 371 Date: Dec. 5, 2007

(51) Int. Cl. C07K 14/435

(52) U.S. Cl. 530/300

(57) ABSTRACT

The present invention provides a novel Kunitz-type inhibitor protein exhibiting high potency to inhibit serine protease activity of human blood coagulation factor Xa. The Kunitz-type inhibitor protein against human blood coagulation factor Xa according to the present invention is
a Kunitz-type inhibitor protein: bitoran D from Bitis arietans venom having the following amino-acid sequence I:

(SEQ ID NO:1)

SKKRP DFCYL PADDG PCRAF IPSFY YNSTS NECNT FIYGG
CYGNA NKFES MDECR KTCVA SA,

a Kunitz-type inhibitor protein: bitoran V from Bitis arietans venom having the following amino-acid sequence II:

(SEQ ID NO:2)

C RQNRP DFCYL PAVEG PCRAY IRSFF YNSTS NECEK FFYGG
CYGNA NKFET RDECR KTCVA SA,

or a modified protein thereof.

### TECHNICAL FIELD

The present invention relates to bitoran which is a trypsin-inhibitor-like protein from venom of Bitis arietans, and its use as an inhibitor against blood coagulation factor Xa. In particular, it relates to bitoran D and bitoran V which are trypsin-inhibitor-like proteins isolated from the venom of Bitis arietans and their modified proteins, as well as their used as an inhibitor specific to blood coagulation factor Xa.

### BACKGROUND ART

In a direct sense, formation of a fibrin clot is a phenomenon initiated by such a direct cause that a α-chain (fibrinopeptide) and β-chain (fibrinofibrinopeptide A) composing fibrinogen are cleaved by the action of thrombin into small peptide fragments (fibrin monomers) and then the resulted peptide fragments (fibrin monomers) are polymerized to form an insoluble fibrin (fibrin multimer). At the same time, thrombin causes activation of factor XIIIa, and the activated enzyme: factor XIIIa catalyzes forming a covalent bond between fibrin molecules, to crosslink fibrin molecules, resulting in a nondegradable clot.

In practice, before formation of said insoluble fibrin (fibrin multimer) caused by the action of thrombin, there exist in blood a process of forming pro-thrombin activator and a process of dividing pro-thrombin (thrombin precursor) by the serine protease activity of the pro-thrombin activator into two fragments, one of which is thrombin. The pro-thrombin activator is composed of an enzyme protein having serine protease activity: factor Xa, its cofactor: factor Va, and a coagulation promoting lipid. The process of conversion of its pro-enzyme factor X into factor Xa is believed to be a mechanism which is directly activated by a factor VIIa/tissue factor complex and indirectly activated by a factor IXa/factor VIIIa phospholipid complex. The serine protease activity of factor Xa is endopeptidase activity for cleaving a peptide chain at C-terminal side of basic amino acid residues, lysine and arginine contained in the peptide chain. In other words, factor Xa has a function comparable to trypsin, a typical serine protease contained in pancreatic fluid.

When factor Xa is produced in a high level in plasma, it begins to bind to a protease inhibitor; TFPI (Tissue Factor Pathway Inhibitor; extrinsic pathway inhibitor (EPI) or Lipoprotein associated coagulation inhibitor (LACI)) present in the plasma. As a result, when a EPI/Xa(LCAI/Xa) complex thus produced further binds on a tissue factor, it becomes a tetramer type of factor VIIa/tissue factor/EPI/Xa (LCAI/Xa) complex, in which enzymatic activity of factor Xa itself is inhibited.

In addition to factor Xa described above, factor VIIa is also an enzyme protein having serine protease activity and selectively binds to such an activated enzyme protein, and there has been observed the presence of an endogenous protease inhibitor; TFPI which acts as a protease inhibitor to the factor. An endogenous protease inhibitor; TFPI specific to human factor Xa is referred to as Kunitz II while an endogenous protease inhibitor; TFPI specific to human factor VIIa is referred to as Kunitz I, and both have been observed to also exhibit inhibitory activity to trypsin from bovine pancreatic fluid. Furthermore, human plasma contains a third endogenous protease inhibitor; TFPI, referred to as Kunitz III, which also exhibits inhibitory activity against trypsin from bovine pancreatic fluid.

Furthermore, structural analysis has been conducted for a proteinic inhibitor: BPTI which inhibits trypsin derived from bovine pancreatic fluid, and it has been found that it has three intramolecular Cys-Cys bonds (S—S bonds) to give a three-dimensional structure, which bonds to an active site of trypsin at the region of Cys-Lys-Ala-Arg-Ile (SEQ ID NO:9) (P₂-P₁-P₁′-P₂′-P₃′). Comparing the amino-acid sequences of the above endogenous protease inhibitors; TFPI, Kunitz I, Kunitz II and Kunitz III with that of the above trypsin inhibitor (BPTI), it may be assumed that these endogenous protease inhibitors; TFPI also have Cys capable of forming an intramolecular Cys-Cys bond (S—S bond) at a corresponding position and three Cys-Cys bonds (S—S bonds) are formed to give a three-dimensional structure.

### DISCLOSURE OF THE INVENTION

### Problem to be Solved by the Invention

In the process of forming a fibrin clot explained above, its direct cause is cleavage of α-chain (fibrinopeptide A) and β-chain (fibrinofibrinopeptide A) composing fibrinogen by the action of thrombin. Thus, the fibrin clot formation can be blocked by inhibition of the enzyme activity of thrombin. Antithrombin III is a known plasmic protease inhibitor that is directly involved in enzyme activity inhibition of thrombin. When heparin is administered in blood, the inhibitor activity of antithrombin III is significantly improved by conversion of a slow-acting enzyme inhibitor to a quick-acting enzyme inhibitor.

Meanwhile, since thrombin is produced as an activated enzyme protein after cleaving pro-thrombin (thrombin precursor) into two fragments by the serine protease activity of a pro-thrombin activator, the thrombin production can be prevented by inhibiting serine protease activity of the pro-thrombin activator, resulting in prevention of fibrin clot formation. In this light, an inhibitor against the serine protease activity of the pro-thrombin activator, specifically, an inhibitor against the serine protease activity of factor Xa, which is a main component for the serine protease activity of the pro-thrombin activator, is also fit to the medical use for preventing fibrin clot formation.

An endogenous plasmic protease inhibitor; TFPI Kunitz II, which has function for inhibiting serine protease activity of factor Xa, has an amino-acid sequence homologous to that of a typical trypsin inhibitor: BPTI, and presumably has a similar three-dimensional structure to that of BPTI. In other words, Cys¹⁷-Arg¹⁸-Gly¹⁹-Tyr²⁰-Ile²¹ (SEQ ID NO:10) for TFPI Kunitz II is corresponding to the partial sequence (P₂-P₁-P₁′-P₂′-P₃′) of the inhibitory activity region; Cys¹⁴-Lys¹⁵-Ala¹⁶-Arg¹⁷-Ile¹⁸ (SEQ ID NO:9) for the trypsin inhibitor: BPTI, and thus the TFPI Kunitz II presumably binds to the serine protease activity center in factor Xa with use of the region, so as to achieve inhibition.

A novel protease inhibitor protein which is not originated from plasma and has specific and higher inhibitory ability to serine protease activity of factor Xa may be a new candidate for such a protein with medical use in the light of medical applications for preventing fibrin clot formation.

To solve the above problems, an objective of the present invention is to provide a novel protease inhibitor protein which is not originated from plasma and has higher inhibitory ability against the serine protease activity of factor Xa.

### Means for Solving Problem

We have focused attention to the fact that plurality of Kunitz type proteins, which are predicted to have a three-dimensional structure similar to that of an endogenous plasmic protease inhibitor such as TFPI Kunitz II having capability of inhibiting serine protease activity of factor Xa, are present in a venom produced by a variety of snakes, and thus have attempted to find a novel Kunitz type protein from a venom which possesses inhibitory effect on serine protease activity of factor Xa. As a result of the search, we have found that a substance exhibiting inhibitory activity to serine protease activity of factor Xa is contained in venom from Bitis arietans. Furthermore, we have attempted to separate the inhibitor against factor Xa which is contained in venom from Bitis arietans with the use of inhibitory activity to serine protease activity of factor Xa as an indicator. As a result, we have achieved such success that a water-soluble protein having an apparent molecular weight of 11 kDa as determined by SDS-PAGE analysis was separated from the venom as a component exhibiting inhibitory activity to serine protease activity of factor Xa. The protein from the venom of Bitis arietans having an apparent molecular weight of 11 kDa, which protein has inhibitory activity to factor Xa, was named as “bitoran”. Furthermore, we have analyzed the amino-acid sequence of the bitoran protein using a purified sample of the bitoran protein having factor Xa inhibiting activity prepared by separation and purification from a crude venom of Bitis arietans arietans, and have revealed that there are actually mixed two proteins having quite similar amino-acid sequences in the purified sample. In the light of characteristic difference in the amino-acid sequences, one is designated as “bitoran D” and the other as “bitoran V”.

Furthermore, comparing the amino-acid sequences of “bitoran D” protein and “bitoran V” protein with the amino-acid sequence of the typical trypsin inhibitor: BPTI, there has been found considerably high level of homology therebetween, and thereby, three-dimensional structures of these proteins are supposed to be at least similar to that of BPTI. On the basis of those findings, we have inferred an inhibitory activity region of the bitoran D and bitoran V proteins, which causes their inhibiting activity specific to factor Xa. We have also found that it is possible to partially modify the natural amino-acid sequence without influencing the configuration of the inferred inhibitory activity region or formation of the overall three-dimensional structure, that is, retaining inhibitory activity of the bitoran D protein and bitoran V protein, which causes their inhibitory activity specific to factor Xa. In other words, we have also found a design strategy for a modified protein from the bitoran D and bitoran V proteins, which modified protein can exhibit inhibitory activity comparable to the factor Xa inhibitory activity of the bitoran D and bitoran V proteins.

Based on the set of findings, we have brought the present invention to completion.

Bitoran D protein according to the present invention is a Kunitz type trypsin-inhibitor-like protein exhibiting high inhibitory potency against the serine protease activity of human blood coagulation factor Xa, which protein is contained in Bitis arietans venom,

wherein the protein is a Kunitz type protein having the following amino-acid sequence I:

(SEQ ID NO:1)

| | |
|---|---|
| S K K R P D F C Y L P A D D G P C R A F | 20 |
| I P S F Y Y N S T S N E C N T F I Y G G | 40 |
| C Y G N A N K F E S M D E C R K T C V A | 60 |
| S A | 62. |

Bitoran V protein according to the present invention is also a Kunitz type trypsin-inhibitor-like protein exhibiting high inhibitory potency against the serine protease activity of human blood coagulation factor Xa, which protein is contained in Bitis arietans venom,

wherein the protein is a Kunitz type protein having the following amino-acid sequence II:

(SEQ ID NO:2)

| | |
|---|---|
| C R Q N R P D F C Y L P A V E G P C R A | 20 |
| Y I R S F F Y N S T S N E C E K F F Y G | 40 |
| G C Y G N A N K F E T R D E C R K T C V | 60 |
| A S A | 63. |

Further, an embodiment of bitoran D protein variant according to the present invention is

a variant of the bitoran D protein being a Kunitz-type trypsin-inhibitor-like protein showing high inhibitory potency against serine protease activity of human blood coagulation factor Xa, which protein is contained in Bitis arietans venom,

wherein the variant protein is a Kunitz-type protein having the following amino-acid sequence III:

(SEQ ID NO:3)

| | |
|---|---|
| S K K R P D F C Y L P A D D G P C R A Y | 20 |
| I P S F Y Y N S T S N E C N T F I Y G G | 40 |
| C Y G N A N K F E S M D E C R K T C V A | 60 |
| S A T R R P T | 67. |

In addition, a modified protein from the bitoran D protein according to the present invention is defined in the following generalized form:

a modified protein derived from the bitoran D being a Kunitz-type trypsin-inhibitor-like protein showing high inhibitory potency against serine protease activity of human blood coagulation factor Xa, which protein is contained in Bitis arietans venom,

wherein the modified protein is a Kunitz-type protein having the following amino-acid sequence IV:

(SEQ ID NO:7)

| | |
|---|---|
| S K K R P D F C Y L P A X1 X2 G P C R A X3 | 20 |
| I X4 S F X5 Y N S T S N E C X6 X7 F X8 Y G G | 40 |
| G C Y G N A N K F E X9 X10 D E C R K T C V | 60 |
| S A | 62 |

In which

X1=D or V; X2=D or E; X3=F or Y; X4=P or R; X5=Y or F;

X6=N or E; X7=T or K; X8=I or F; X9=S or T; X10=M or R.

Similarly, a modified protein form the bitoran V protein according to the present invention is defined in the following generalized form:

a modified protein derived from the bitoran V being a Kunitz-type trypsin-inhibitor-like protein showing high inhibitory potency against serine protease activity of human blood coagulation factor Xa, which protein is contained in Bitis arietans venom,

wherein the modified protein is a Kunitz-type protein having the following amino-acid sequence V:

(SEQ ID NO:8)

| | |
|---|---|
| C R Q N R P D F C Y L P A X1 X2 G P C R A | 20 |
| X3 I X4 S F X5 Y N S T S N E C X6 X7 F X8 Y G | 40 |
| G C Y G N A N K F E X9 X10 D E C R K T C V | 60 |
| A S A | 63 |

In which

X1=D or V; X2=D or E; X3=F or Y; X4=P or R; X5=Y or F;

X6=N or E; X7=T or K; X8=I or F; X9=S or T; X10=M or R.

In addition, the present invention provides a use invention for the afore-mentioned bitoran proteins or modified protein thereof according to the present invention,

specifically, the use invention according to the present invention is:

use of the bitoran protein or modified protein thereof as defined above for preparing an inhibitary composition against human blood coagulation factor Xa, wherein the bitoran protein or modified protein thereof is used as an active ingredient having inhibitory activity to serine protease activity of human blood coagulation factor Xa.

### Effect of the Invention

The bitoran protein from Bitis arietans venom according to the present invention, particularly bitoran D protein or bitoran V protein, is one of Kunitz-type trypsin-inhibitor-like proteins, and shows high inhibitory potency, among others, against serine protease activity of human blood coagulation factor Xa, so that the protein can be suitably used as an inhibitor specific to human blood coagulation factor Xa, which is available for controlling the step of forming thrombin from pro-thrombin (thrombin precursor) that is just an initial stage in a process for fibrin clot formation.

### BRIEF DESCRIPTION OF THE DRAWINGS

FIG. 1 is a chart showing elution fractions (indicated by a horizontal bar) in the reverse phase HPLC column purification step for bitoran protein having high inhibitory activity to serine protease activity of blood coagulation factor Xa, which bitoran protein was separated and purified from a crude Bitis arietans arietans venom purchased from LATOXAN, and SDS-PAGE analysis results (R: reduced state, NR: non-reduced state) for the purified bitoran protein.

FIG. 2 is a chart showing elution fractions (indicated by a horizontal bar) in the reverse phase HPLC column purification step for bitoran protein having high inhibitory activity to serine protease activity of blood coagulation factor Xa, which bitoran protein was separated and purified from crude venom of Bitis arietans obtained from the Japan Snake Institute, and SDS-PAGE analysis results (R: reduced state, NR: non-reduced state) for the purified bitoran protein.

FIG. 3 is a drawing showing comparison between the amino-acid sequences of two bitoran proteins; bitoran D (SEQ ID NO:1) and bitoran V (SEQ ID NO:2) separated and purified from crude venom of Bitis arietans arietans purchased from LATOXAN, as well as three Cys-Cys bonds present in their three-dimensional structures and a site for modification with a sugar chain. In this figure, “—CHO” indicates a site for modification with a sugar chain, and “→” indicates a site in an inhibitory activity region, which corresponds to a cleavage site for a trypsin type serine protease.

FIG. 4 is a drawing showing comparison between the evaluation results for inhibitory activity against serine protease activity of blood coagulation factor Xa of a bitoran protein (a mixture of bitoran D and bitoran V), which was separated and purified from a crude Bitis arietans arietans venom purchased from LATOXAN and for inhibitory activity against serine protease activity of blood coagulation factor Xa exhibited by SBTI (Kunitz-type trypsin inhibitor from soybean).

FIG. 5 is a drawing showing comparison among the amino-acid sequences of Kunitz type mature proteins and precursor proteins contained in venoms from various snakes (Bitis gabonica, SEQ ID NOs:18 and 19; Vipera ammodytes ammodytes, SEQ ID NO:20; Vipera ammodytes, SEQ ID NO:21; Oxyuranus scutellatus scutellatus, SEQ ID NO:22; Eristocophis macmahonii, SEQ ID NO:23; Hemachatus haemachatus, SEQ ID NO:24; Daboia russellii siamensis, SEQ ID NO:25; Naja nivea, SEQ ID NO:26; Tropidechis carinatus, SEQ ID NO:27; Notechis scutatus scutatus, SEQ ID NO:28; Pseudonaja textiles textiles, SEQ ID NOs:29 and 35; Oxyuranus microlepidotus, SEQ ID NO:30, Pseudechis australis, SEQ ID NO:31; Oxyuranus scutellatus, SEQ ID NO:32; Ophiophagus hannah, SEQ ID NO:33; Dendroaspis polylepis polylepis, SEQ ID NO:34; Bungarus candidus, SEQ ID NO:36; Dendroaspis angusticeps, SEQ ID NO:37) which have trypsin inhibitor activity, and Kunit-type trypsin-inhibitor-like proteins according to the present invention; bitoran D (SEQ ID NO:1), bitoran V (SEQ ID NO:2) and a bitoran precursor protein contained in the venom from Bitis arietans, which exhibit inhibitory activity against blood coagulation factor Xa (SEQ ID NO:5).

FIG. 6 is drawing showing the nucleotide sequence of a cDNA encoding a bitoran precursor protein, (SEQ ID NO:4) which was cloned from cDNA library prepared from all mRNA extracted from a poison gland of Bitis arietans purchased from SAV, and the amino-acid sequence encoded in the nucleotide sequence of its ORF.

FIG. 7 is a drawing showing comparison between the amino-acid sequences of two bitoran proteins; bitoran D (SEQ ID NO:1) and bitoran V (SEQ ID NO:2) separated and purified from a crude venom of Bitis arietans arietans purchased from LATOXAN, and the amino-acid sequence encoded in the nucleotide sequence of an ORF in the cDNA encoding a bitoran precursor protein cloned from a poison gland of Bitis arietans purchased from SAV (SEQ ID NO:5).

FIG. 8 is a drawing showing comparison between partial amino-acid sequences of inhibitory activity regions found in Kunitz-type trypsin-inhibitor-like proteins from various organisms (Vipera ammodytes ammodytes, SEQ ID NO:40; Vipera ammodytes, SEQ ID NO:41; Pseudonuja textiles textilis, SEQ ID NO:42; Daboia russellii siamensis, SEQ ID NO:43; Hemachatus haemachatus, SEQ ID NO:44; Naja naja naja, SEQ ID NO:45; Ophiophagus hannah, SEQ ID NO:46; Dendoroaspis polylepis polylepis, SEQ ID NO:47; Bungarus fasciatus, SEQ ID NO:48; Dendroaspis angusticeps, SEQ ID NO:49; Eristocophis macmahonii, SEQ ID NO:50; Naja nivea, SEQ ID NO:51; Oryctolagus cuniculus, SEQ ID NO:52; Homo sapiens, SEQ ID NOs:53, 54, 55 and 58; Xenopus laevis, SEQ ID NO:56; Mus musculus, SEQ ID NO:57; Equus caballus, SEQ ID NO:59), and partial amino-acid sequences of inhibitory activity regions contained in the Kunitz-type trypsin-inhibitor-like proteins according to the present invention; bitoran D (SEQ ID NO:1) and bitoran V (SEQ ID NO:2) contained in venom from Bitis arietans.

### BEST MODE FOR CARRYING OUT THE INVENTION

There will be more specifically explained the present invention.

As described in Examples later, a Kunitz type trypsin-inhibitor-like protein; bitoran according to the present invention is isolated from venom of Bitis arietans. Specifically, bitoran proteins that were separated and purified from crude venom of Bitis arietans arietans purchased from LATOXAN are bitoran D protein having the following amino-acid sequence I:

(SEQ ID NO:1)

| | |
|---|---|
| S K K R P D F C Y L P A D D G P C R A F | 20 |
| I P S F Y Y N S T S N E C N T F I Y G G | 40 |
| C Y G N A N K F E S M D E C R K T C V A | 60 |
| S A | 62. |

and, bitoran V protein having the following amino-acid sequence II:

(SEQ ID NO:2)

| | |
|---|---|
| C R Q N R P D F C Y L P A V E G P C R A | 20 |
| Y I R S F F Y N S T S N E C E K F F Y G | 40 |
| G C Y G N A N K F E T R D E C R K T C V | 60 |
| A S A | 63. |

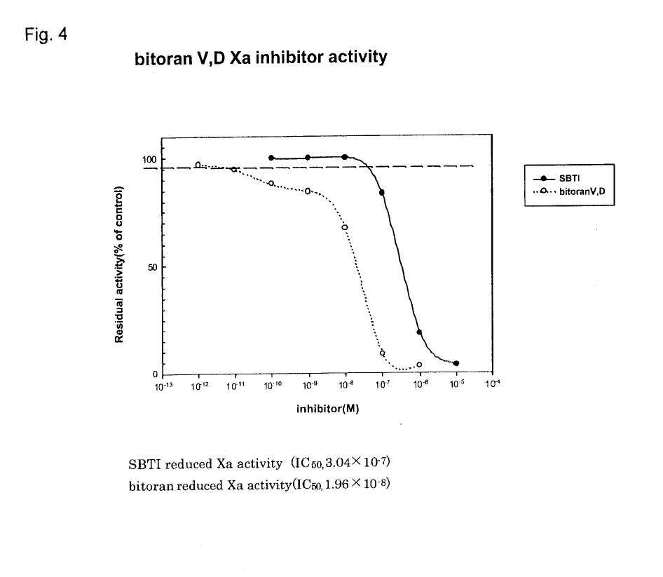

The bitoran V protein was also separated and purified from a venom obtained from the Japan Snake Institute. Both the proteins are protein components contained in the venom, each of which is secreted as a mature protein from poison gland cells after its portion of N-termini has been truncated by post-translation processing. Bitoran D protein and bitoran V protein, which are contained in the venoms from Bitis arietans, exhibit high inhibitory potency against the serine protease activity of human blood coagulation factor Xa, as shown in FIG. 4.

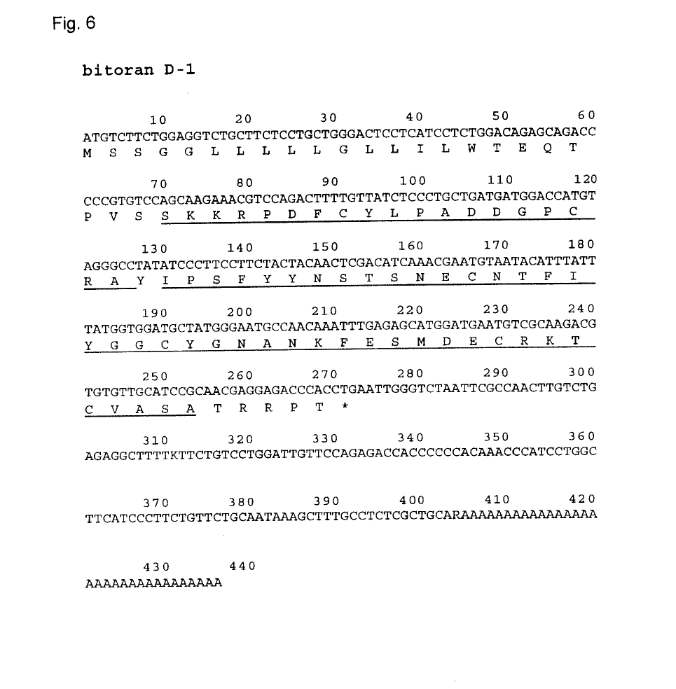

In addition, we extracted mRNAs including an mRNA used in translation of a bitoran precursor protein, from which the mature protein is derived, from a freeze-dried poison gland of Bitis arietans purchased from SAV Inc., South Africa, to prepare cDNA thereof. When cloning was made on the cDNA, cloned was a cDNA having the nucleotide sequence shown in FIG. 6 (SEQ ID No. 4), which encodes the following amino-acid sequence VI:

(SEQ ID NO:5)

| | |
|---|---|
| M S S G G L L L L L G L L I L W T E Q T P V S | 23 |
| S K K R P D F C Y L P A D D G P C R A Y | 43 |
| I P S F Y Y N S T S N E C N T F I Y G G | 63 |
| C Y G N A N K F E S M D E C R K T C V A | 83 |
| S A T R R P T | 90. |

Comparing the amino-acid sequence VI (SEQ ID No. 5) with the amino-acid sequence I (SEQ ID No. 1) and the amino-acid sequence II (SEQ ID No. 2), there is found very high homology between the amino-acid sequence VI (SEQ ID No. 5) and the amino-acid sequence I (SEQ ID No. 1), and thus the amino-acid sequence VI (SEQ ID No. 5) is concluded to be the amino-acid sequence of a precursor protein for such a mature protein being equivalent to the bitoran D protein. The mature bitoran protein to be obtained from the precursor protein having the amino-acid sequence VI (SEQ ID No. 5) is designated as “bitoran D′”. The bitoran D′ protein is considered to be secreted as a mature protein having the following amino-acid sequence III:

(SEQ ID NO:3)

| | |
|---|---|
| S K K R P D F C Y L P A D D G P C R A Y | 20 |
| I P S F Y Y N S T S N E C N T F I Y G G | 40 |
| C Y G N A N K F E S M D E C R K T C V A | 60 |
| S A T R R P T | 67 |

after its N-terminus is truncated by post-translation processing in quite similar manner to the bitoran D protein.

The physiological function of the bitoran D′ protein is considered to be equivalent to those of the bitoran D protein and bitoran V protein. In other words, the bitoran D′ protein is also believed to be a Kunitz-type trypsin-inhibitor-like protein having high inhibitory potency against the serine protease activity of human blood coagulation factor Xa.

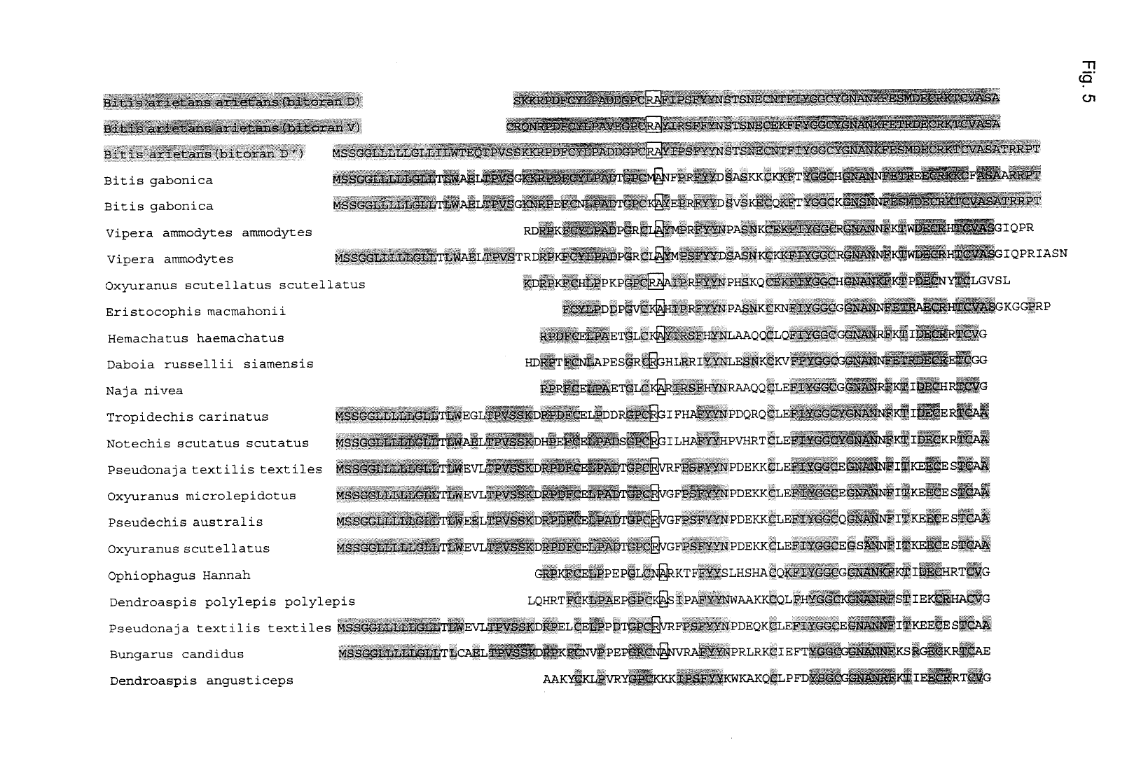

Comparing the amino-acid sequences of the bitoran D protein, bitoran V protein contained in the venom and the precursor of the bitoran D′ protein form Bitis arietans with the amino-acid sequences of Kunitz-type trypsin-inhibitor-like proteins contained in various venoms or their precursors which have been already reported, there can be found considerable homology as shown in FIG. 5, and thus it is understood that they probably constitute one family. In terms of the amino-acid sequence of the above bitoran D′ protein precursor, the underlined part in the N-terminus seems to be very similar to the corresponding amino-acid sequence found even in a precursor for a Kunitz-type trypsin-inhibitor-like protein contained in another venom, and therefore its N-terminus is concluded to be truncated by post-translation processing when the protein is secreted as a mature protein.

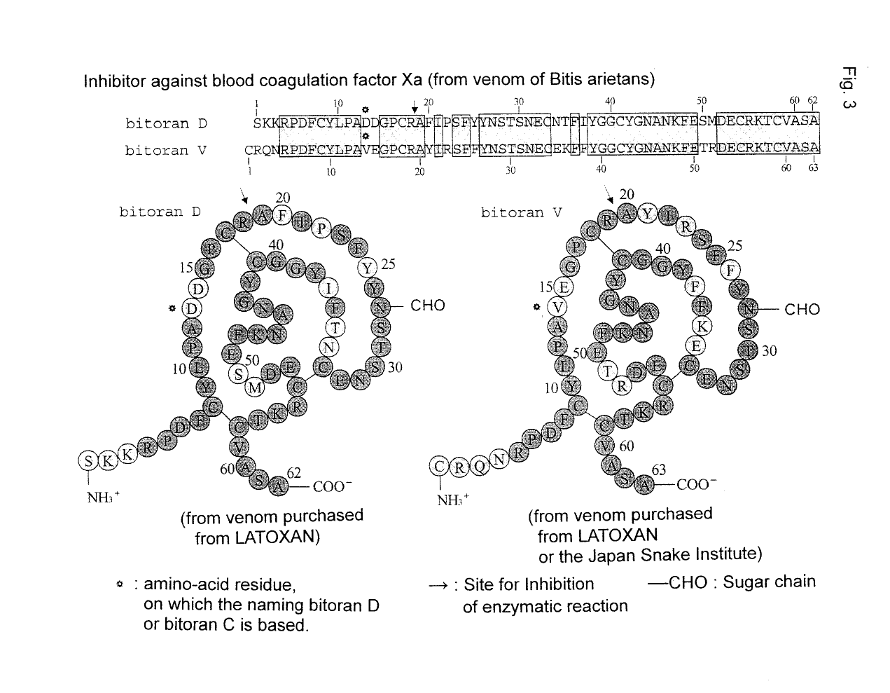

For the bitoran D protein or bitoran V protein being one of Kunitz-type trypsin-inhibitor-like proteins, when being secreted, the N-terminus of its precursor protein is truncated by post-translation processing, and then intramolecular Cys-Cys bonds are formed therein, whereby the resulted protein has a similar three-dimensional structure to that of the typical trypsin inhibitor: BPTI. Specifically, each of the protein folds into such a structure having intramolecular Cys-Cys bonds as schematically shown in FIG. 3, which has a modification with a sugar chain on the specific Asn residue. As for an inhibitory activity region having higher inhibitory potency against the serine protease activity of human blood coagulation factor Xa, which corresponds to the region of Cys¹⁴-Lys¹⁵-Ala¹⁶-Arg¹⁷-Ile¹⁸ (SEQ ID NO:9) (P₂-P₁-P₁-P₂′-P₃′) for the proteinic inhibitor: BPTI presumably involved in binding to an active region in trypsin, Cys¹⁷-Arg¹⁸-Ala¹⁹-Phe²⁰-Ile²¹ (SEQ ID NO:11) and Cys¹⁸-Arg¹⁹-Ala²⁰-Tyr²¹-Ile²² (SEQ ID NO:12) are identified in the bitoran D protein and bitoran V protein, respectively.

The above-described inhibitory activity region in the bitoran protein seems to be retained in a configuration permitting a suitable binding to human blood coagulation factor Xa by its whole three-dimensional structure. Specifically, In similar manner to the intramolecular Cys-Cys bonds formed at the three sites of [Cys⁵:Cys⁵⁵], [Cys¹⁴:Cys³⁸] and [Cys³⁰:Cys⁵¹], which are essential for folding up the three-dimensional structure of the trypsin inhibitor (BPTI), the formation of three intramolecular Cys-Cys bonds at [Cys⁸:Cys⁵⁸], [Cys¹⁷:Cys⁴¹] and [Cys³³:Cys⁵⁴] in the bitoran D protein and at [Cys⁹:Cys⁵⁶], [Cys¹⁸:Cys⁴²] and [Cys³⁴:Cys⁵⁵] in the bitoran V protein define the whole three-dimensional structure thereof. These three intramolecular Cys-Cys bonds (S—S bonds) are formed as follows; at first, a peptide chain takes a configuration suitable for these three intramolecular Cys-Cys bonds, and then —S—S— bonds are formed between —SHs on adjacent Cys residues.

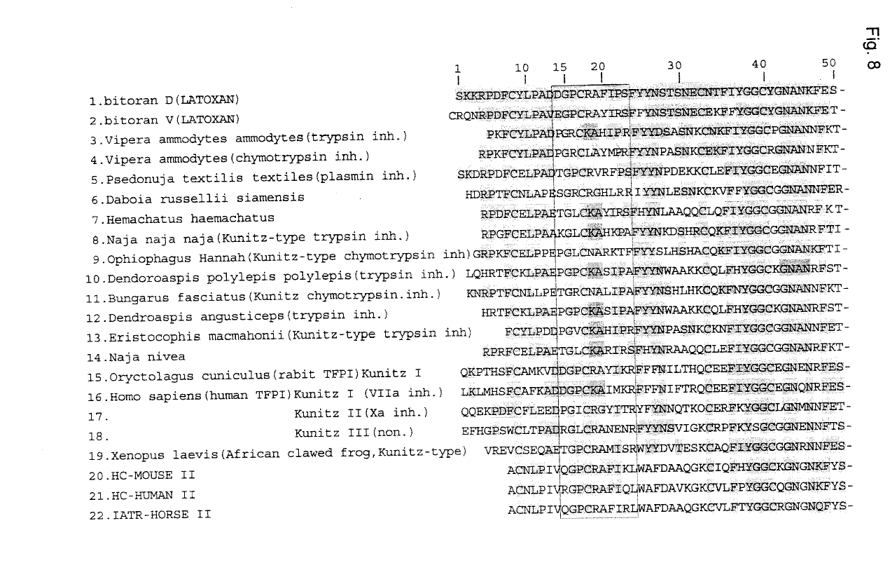

Indeed, comparing the partial amino-acid sequences of inhibitory activity regions of the bitoran D protein and bitoran V protein with those of Kunitz-type trypsin-inhibitor-like proteins from various organisms, the positions of Cys's used in the three intramolecular Cys-Cys bonds are well conserved as shown in FIG. 8. In contrast, comparing the partial amino-acid sequences of peptide chains having inhibitory activity regions, that is, the regions between Cys¹⁷ to Cys³⁰ in the bitoran D protein and between Cys¹⁸ to Cys³¹ in the bitoran V protein, there is not very high homology. Although the whole three-dimensional structures are quite similar, such difference in a partial amino-acid sequence influences a detailed configuration of the inhibitory activity region in each of the Kunitz-type trypsin-inhibitor-like proteins, and thus, is probably a determinant factor of its inhibitory properties. Similar presumption may be made for comparison of the amino-acid sequences of the bitoran D protein, bitoran V protein and bitoran D′ protein precursor shown in FIG. 5 with the amino-acid sequences of Kunitz-type trypsin-inhibitor-like proteins or their precursors contained in various venoms which have been already reported.

In other words, it may be concluded that the three-dimensional structures of the bitoran D protein and bitoran V protein can be retained by essentially conserving, in amino-acid sequences of the bitoran D protein, bitoran V protein and bitoran D′ protein precursor, the regions having very high homology, that is, the regions of Arg³ to Ala⁶² in the bitoran D protein and of Arg⁴ to Ala⁶³ in the bitoran V protein, and thus, the configuration of the inhibitory activity region can be retained such that an appropriate binding can be made to human blood coagulation factor Xa. Specifically, it may be concluded that a modified protein having the following amino-acid sequence VII as the partial amino-acid sequence of such a region:

(SEQ ID NO:13)

R P D F C Y L P A X1 X2 G P C R A X3
I X4 S F X5 Y N S T S N F C X6 X7 F X8 Y G G
C Y G N A N K F E X9 X10 D E C R K T C V A
S A

in which

X1=D or V; X2=D or E; X3=F or Y; X4=P or R; X5=Y or F;

X6=N or E; X7=T or K; X8=I or F; X9=S or T; X10=M or R,

is to be a Kunitz-type trypsin-inhibitor-like protein having as high inhibitory potency against the serine protease activity of human blood coagulation factor Xa as that of the bitoran D protein and bitoran V protein.

A modified protein from the bitoran protein according to the present invention retains the above amino-acid sequence VII. In particular, a modified protein from the bitoran D protein according to the present invention is a Kunitz type protein having the following amino-acid sequence IV:

(SEQ ID NO:7)

| | |
|---|---|
| S K K R P D F C Y L P A X1 X2 G P C R A X3 | 20 |
| I X4 S F X5 Y N S T S N E C X6 X7 F X8 Y G G | 40 |
| G C Y G N A N K F E X9 X10 D E C R K T C V | 60 |
| S A | 62 |

wherein

X1=D or V; X2=D or E; X3=F or Y; X4=P or R; X5=Y or F;

X6=N or E; X7=T or K; X8=I or F; X9=S or T; X10=M or R.

Similarly, a modified protein from the bitoran V protein according to the present invention is a Kunitz type protein having the following amino-acid sequence V:

(SEQ ID NO:8)

| | |
|---|---|
| C R Q N R P D F C Y L P A X1 X2 G P C R A | 20 |
| X3 I X4 S F X5 Y N S T S N E C X6 X7 F X8 Y G | 40 |
| G C Y G N A N K F E X9 X10 D E C R K T C V | 60 |
| A S A | 63 |

in which

X1=D or V; X2=D or E; X3=F or Y; X4=P or R; X5=Y or F;

X6=N or E; X7=T or K; X8=I or F; X9=S or T; X10=M or R.

A bitoran protein according to the present invention probably has the same topological configuration as that of the proteinic inhibitor: BPTI. Therefore, the inhibitory activity region in the bitoran protein presumably involved in binding to human blood coagulation factor Xa is a partial structure formed of the partial amino-acid sequence of -Gly¹⁵-Pro¹⁶-Cys¹⁷-Arg¹⁸-Ala¹⁹-Phe²⁰-Ile²¹-Pro²²-Ser²³- . . . -Cys³³ (SEQ ID NO:14) in the bitoran D protein or of -Gly¹⁶-Pro¹⁷-Cys¹⁸-Arg¹⁹-Ala²⁰-Tyr²¹-Ile²²-Arg²³-Ser²⁴- . . . -Cys³⁴ (SEQ ID NO:15) in the bitoran V protein. Meanwhile, the N-terminal partial amino-acid sequence and the C-terminal partial amino-acid sequence in the above partial amino-acid sequence, which is involved in forming three intramolecular Cys-Cys bonds, which determine the whole configuration of the bitoran protein, have considerably high similarity to each of the other Kunitz-type trypsin-inhibitor-like proteins, as seen in amino-acid sequence alignments shown in FIGS. 5 and 8. A configuration of an inhibitory activity region probably involved in binding to human blood coagulation factor Xa may be, therefore, also retained in a chimeric protein where the partial amino-acid sequence of -Gly¹⁵-Pro¹⁶-Cys¹⁷-Arg¹⁸-Ala¹⁹-Phe²⁰-Ile²¹-Pro²²-Ser²³- . . . -Cys³³ (SEQ ID NO:14) in the bitoran D protein or of -Gly¹⁶ Pro¹⁷-Cys¹⁸-Arg¹⁹-Ala²⁰-Tyr²¹-Ile²²-Arg²³-Ser²⁴- . . . -Cys³⁴ (SEQ ID NO:15) in the bitoran V protein are linked with the N-terminal partial amino-acid sequence and the C-terminal partial amino-acid sequence in the above partial amino-acid sequence derived from each of the other Kunitz-type trypsin-inhibitor-like proteins. However, in terms of two intramolecular Cys-Cys bonds at [Cys¹⁷:Cys⁴¹] and [Cys³³:Cys⁵⁴] in the bitoran D protein or two intramolecular Cys-Cys bonds at [Cys¹⁸:Cys⁴²] and [Cys³⁴:Cys⁵⁵] in the bitoran V protein, an efficiency for forming them (holding efficiency) in the chimeric protein is not necessarily equivalent to that in the original bitoran protein. On the other hand, in the case where the amino-acid sequence VII is chosen, an efficiency for forming the whole three-dimensional structure (holding efficiency) is probably equivalent to that in the original bitoran protein, and thus such selection is more preferable for the modified protein.

Additionally, in terms of a partial amino-acid sequence corresponding to an inhibitory activity region, by choosing G P C R A X3 I X4 X11 X12 X13 X14 N S T S N E C (SEQ ID NO:16) wherein X3=F or Y; X4=P or R; X11=A or S; X12=F or Y; X13=F or Y; X14=F or Y, a partial structure equivalent to the original bitoran protein can be retained, and therefore, inhibitory activity to human blood coagulation factor Xa is predicted to be retained.

A modified protein from the bitoran protein according to the present invention may be prepared by the process comprising the steps of inducing mutation in a gene encoding the amino-acid sequence of the aforementioned precursor of the bitoran D′ protein to form a gene encoding such a modified protein from the bitoran protein, and then expressing it to produce the protein as a recombinant protein. In the process for preparing the recombinant protein, a suitably used expression system may be, for example, an expression system used for recombinant expression of a Kunitz-type trypsin-inhibitor-like protein such as the trypsin inhibitor (BPTI). In such expression, a desirable leader sequence or signal sequence in the N-terminus to be truncated during forming a mature protein may be appropriately selected, depending on an expression system employed.

A transformed strain (bitoran D/XL-1) obtained by introducing, into a host E. coli strain XL-1 Blue, a vector: bitoran D-PGEM T-easy in which a gene (cDNA: SEQ ID No. 4) encoding the precursor protein of the Kunitz-type trypsin-inhibitor-like protein from Bitis arietans; bitoran according to the present invention is inserted into a cloning site in a cloning vector: PGEM T-easy vector is designated as KM1 and has been subjected to international deposit (dated May 13, 2005) as a deposit number FERM BP-10335 to National Institute of Advanced Industrial Science and Technology, International Patent Organism Depositary (Chuo-6, 1-1-1, Higashi, Tsukuba, Ibaragi, Japan, zip 305-8566) under the Budapest Treaty.

When using the bitoran protein or modified protein thereof according to the present invention for preparing an inhibitory composition against human blood coagulation factor Xa as an active ingredient having inhibitory activity against the serine protease activity of human blood coagulation factor Xa, a content of the bitoran protein or modified protein thereof in the composition may be appropriately determined, depending on its inhibitory activity (IC₅₀).

### EXAMPLES

There will be more specifically described the trypsin-inhibitor-like peptide from the venom according to the present invention; bitoran and its pharmacological function.

Isolation of the Trypsin-Inhibitor-Like Peptide from the Venom; Bitoran

We have studied physiological functions of a variety of proteins contained in a venom, and have finally found that a protein inhibiting activity of a trypsin type serine protease is contained therein. Specifically, we have found that there is a protein exhibiting inhibitory effect to the serine protease activity of human blood coagulation factor Xa among various proteins contained in the venom from Bitis arietans.

At first, following the procedure below, a protein component having inhibitory potency against the serine protease activity of human factor Xa was separated from various protein components contained in the venom of Bitis arietans. For the separation, column chromatography was utilized, an apparatus used for column chromatography was FPLC system with AKTA explorer 10S (Amersham Biosciense Inc.), and a liquid temperature was selected at 4° C. A protein component contained in a column eluate was monitored utilizing an absorbance at a wavelength of 280 nm (A280) measured by AKTA explorer 10S, which was used as an index for a protein content.

Crude venom of Bitis arietans arietans purchased from LATOXAN was dissolved in Tris-HCl buffer (50 mM Tris-HCl pH 8.0) to prepare 2 mL of a crude venom solution (in concentration of 250 mg/mL). The crude venom solution was applied to Superdex 200 pg gel permeation column (φ2.6 cm×L 60 cm) equilibrated with the Tris-HCl buffer (50 mM Tris-HCl pH 8.0) at a flow rate of 2 mL/min. Two mL aliquots of the series of eluted fractions were collected, and each eluted fraction was determined for the presence of inhibitory activity against the serine protease activity of blood coagulation factor Xa. In the assay for inhibitory activity specific to the serine protease activity of blood coagulation factor Xa, a synthetic substrate Boc-leucyl-glycil-L-arginyl-p-nitroanilide hydrochloride was used to determine a concentration of a reaction product formed by truncation at the C-terminal side of an arginine residue by protease activity of blood coagulation factor Xa (Cproduct: concentration of p-nitroaniline), and then from a product concentration without addition of a test substance: Cproduct₀ and a product concentration with addition of a test substance: Cproduct, an inhibitory rate was calculated as (Cproduct₀−Cproduct)/Cproduct₀. Fractions exhibiting inhibitory activity in the inhibitory activity assay were pooled.

Next, the pooled inhibitory activity fractions were applied to Q-Sepharose High Performance column (φ1.6 cm×L 60 cm) equilibrated with the same Tris-HCl buffer. Elution of the column was conducted with use of 8 column volume under a linear gradient condition up to 0.7M NaCl at a flow rate of 2 mL/min. Two mL aliquots of eluted fractions were collected and each eluted fraction was determined for the presence of inhibitory activity using the inhibitory activity assay. Inhibitory activity was observed only in the fractions eluted near 0.1 M NaCl. The fractions exhibiting inhibitory activity were collected and lyophilized to collect a protein component.

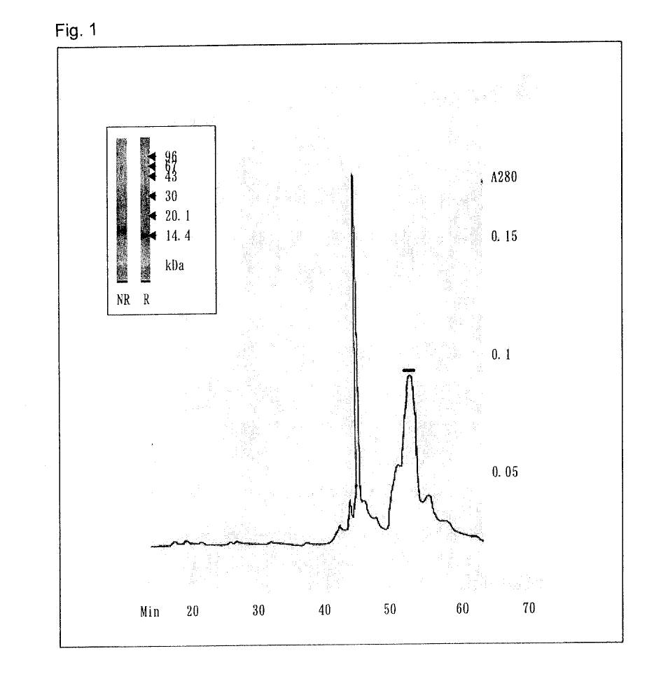

For further purification, the lyophilized protein component was applied to COSMOSIL 5C18 AR-300 column (φ4.6 mm×L 250 mm) for reverse phase HPLC. In FIG. 1, eluted fractions containing the protein component having the protease inhibitory activity in the reverse phase HPLC column purification were indicated by a horizontal bar. The sample purified by the reverse phase HPLC was analyzed in a non-reduced state (NR) and a reduced state (R) by SDS-PAGE. As shown in FIG. 1, the SDS-PAGE analysis gave a single band, but there is a difference in an apparent molecular weight between the non-reduced state (NR) and the reduced state (R), indicating that there is an intramolecular cross-link by Cys-Cys bonding (S—S bond).

The protein having inhibitory activity against the serine protease activity of human blood coagulation factor Xa which was isolated under the aforementioned column purification conditions was designated as “bitoran”. The column purification gave 0.65 mg of purified sample of the lyophilized bitoran protein from 250 mg of the lyophilized crude venom.

Furthermore, isolation of a protein having inhibitory activity to serine protease activity of human blood coagulation factor Xa was also attempted from crude venom of Bitis arietans obtained from the Japan Snake Institute.

In 25 mL of Tris-HCl buffer (50 mM Tris-HCl pH 8.0) was dissolved 500 mg of the crude venom from Bitis arietans to prepare 25 mL of a solution of the crude venom at concentration of 20 mg/mL. Next, the inhibitory activity fraction was applied to Q-Sepharose High Performance column (φ1.6 cm×L 10 cm) equilibrated with the same Tris-HCl buffer. Elution of the column was conducted with 8 column volume under a linear gradient condition up to 0.7M NaCl at a flow rate of 2 mL/min. Two mL aliquots of eluted fractions were collected and each eluted fraction was determined for the presence of inhibitory activity using the aforementioned inhibitory activity assay. Inhibitory activity was observed only in the fractions eluted near 0.1 M NaCl.

After collection, the pooled fractions exhibiting inhibitory activity were concentrated by the use of Amicon 3000 MW cut-off (Millipore). The concentrated sample of partially purified fractions was applied to Superdex 200 pg gel permeation column (φ2.6 cm×L 60 cm) equilibrated with the same Tris-HCl buffer at a flow rate of 2 mL/min. Two mL aliquots of the eluted fractions were collected and each eluted fraction was determined for the presence of inhibitory activity specific to the serine protease activity of blood coagulation factor Xa. The fractions exhibiting inhibitory activity in the inhibitory activity assay were pooled.

The pooled fraction exhibiting inhibitory activity was diluted with an imidazole-HCl buffer (20 mM imidazole-HCl, pH6.0) and then applied to SP-Sepharose High Performance column (φ1.6 cm×L 11 cm). During the elution, a protein component exhibiting inhibitory activity was collected in a flowing-through fraction. The flowing-through fraction was similarly concentrated using Amicon 3000 MW cut-off.

Finally, purification was conducted by applying the concentrated flowing-through fraction to COSMOSIL 5C18 AR-300 column (φ4.6 mm×L 250 mm) for reverse phase HPLC. Elution was conducted under the linear gradient condition of 0.1% aqueous TFA solution/acetonitrile (CH₃CN) with a CH₃CN concentration of 0 to 60% in 240 min.

The protein component exhibiting inhibitory activity was eluted at the position of a horizontal bar shown in FIG. 3. The sample purified by the reverse phase HPLC was analyzed in a non-reduced state (NR) and a reduced state (R) by SDS-PAGE. As shown in FIG. 3, the SDS-PAGE analysis gave a single band, but there is a difference in an apparent molecular weight between the non-reduced state (NR) and the reduced state (R), indicating that there is an intramolecular cross-link by Cys-Cys bonding (S—S bond).

Under the above column purification conditions, 0.7 mg of purified sample of a lyophilized bitoran protein was obtained from 500 mg of the crude venom of Bitis arietans obtained from the Japan Snake Institute.

Amino-Acid Sequence Analysis of the Bitoran

Since the isolated protein component exhibiting protease inhibitory activity has intramolecular Cys-Cys bonding (S—S bond) as described above, the Cys-Cys bonding (S—S bond) was reduced and a sulfanyl group (—SH) on a side chain of Cys residue was subjected to treatment of S-alkylation. Then, the product was again applied to the reverse phase HPLC column for purifying and isolating the S-alkylated peptide. When observing a peak of the S-alkylated peptide eluted from the reverse phase HPLC column, two peaks were found for the lyophilized bitoran protein sample obtained from the crude venom of Bitis arietans arietans purchased from LATOXAN. Each of the S-alkylated peptides showing these two peaks were collected and lyophilized.

The two S-alkylated peptides were subjected to amino-acid analysis by the following procedure.

5 nmol equivalents of the lyophilized S-alkylated peptides (55 μg) were fragmented by enzymatic-digestion at 37° C. for 24 hours using endopeptidases Lys-C, Asp-N and Arg-C. Each peptide fragment obtained by the enzymatic digestion with use of endopeptidases was collected by reverse phase HPLC, and then analyzed for the amino-acid sequence using a protein sequencer (Applied Biosystems 473A, 477, Shimadzu PPSQ-21A). Separately, the lyophilized S-alkylated peptide was desalted and then analyzed for the N-terminal amino-acid sequence thereof by the protein sequencer.

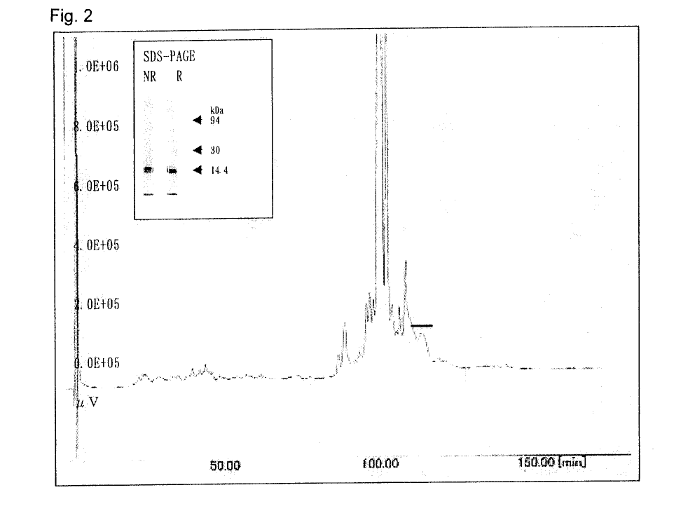

Based on the amino-acid sequence analysis results for the N-terminal amino-acid sequence and the amino-acid sequence for the peptide fragments obtained by each endopeptidase enzyme digestion, the whole amino-acid sequences of the S-alkylated peptides were determined. FIG. 2 shows comparison of the determined whole amino-acid sequences for the S-alkylated peptides showing two peaks. It was found that these amino-acid sequences exhibited quite high homology. The bitoran protein with 62 amino acids was designated as “bitoran D”, while the bitoran protein with 63 amino acids was designated as “bitoran V”. In other words, a peptide fragment obtained by enzyme digestion with endopeptidase Asp-N are definitely different between these, and the difference is due to the fact that the 13th amino acid is D (Asp) in the bitoran D protein, whereas the corresponding 14th amino acid is V (Val) in the bitoran V protein The above designations take the characteristic difference in an amino acid into account.

In the case of the bitoran D protein and bitoran V protein, there were also conserved six Cys's that are expected to be involved in formation of the three of intramolecular Cys-Cys bondings (S—S bonds), which were found in the group of protease inhibitors exhibiting high homology to the trypsin inhibitor (BPTI) such as an endogenous protease inhibitor specific to human factor Xa; TFPI Kunitz II. Specifically, three of intramolecular Cys-Cys bonding (S—S bond), i.e., [Cys⁸:Cys⁵⁸], [Cys¹⁷:Cys⁴¹] and [Cys³³:Cys⁵⁴] in the bitoran D protein and [Cys⁹:Cys⁵⁶], [Cys¹⁸:Cys⁴²] and [Cys³⁴:Cys⁵⁵] in the bitoran V protein, are predicted to be formed in such a manner equivalent to the three intramolecular Cys-Cys bonding; [Cys⁵:Cys⁵⁵], [Cys¹⁴:Cys³⁸] and [Cys³⁰:Cys⁵¹] in the trypsin inhibitor (BPTI) which are essential for forming the three-dimensional structure thereof. The bitoran D protein and bitoran V protein also, therefore, fold in very similar three-dimensional structure to that of the trypsin inhibitor (BPTI) in association with formation of the three intramolecular Cys-Cys bonding (S—S bond) as illustrated in FIG. 3.

Cys¹⁷-Arg¹⁸-Ala¹⁹-Phe²⁰-Ile²¹ (SEQ ID NO:11) in the bitoran D protein and Cys¹⁸-Arg¹⁹-Ala²⁰-Tyr²¹-Ile²² (SEQ ID NO:12) in the bitoran V protein are identified as regions corresponding to the region Cys¹⁴-Lys¹⁵-Ala¹⁶-Arg¹⁷-Ile¹⁸ (SEQ ID NO:9) (P₂—P₁-P₁′-P₂′-P₃′) found in the proteinic inhibitor: BPTI inhibiting trypsin from bovine pancreatic fluid, which region is believed to be a region involved in bonding to an active site of trypsin.

Furthermore, —CO—NH₂ on the side chain of Asn²⁷ residue in the bitoran D protein contained in a natural venom and —CO—NH2 on the side chain of Asn²⁸ residue in the bitoran V protein are identified as a site for modification with a sugar chain by N-glycosylation.

For the sample of lyophilized bitoran protein purified from the crude venom of Bitis arietans obtained from the Japan Snake Institute, only one peak was found when observing a S-alkylated peptide peak eluted from the reverse phase HPLC column. The S-alkylated peptide showing the single peak was collected, lyophilized and subjected to amino-acid analysis as described above. The whole amino-acid sequence determined was substantially identical to that of the bitoran V protein.

Inhibitory Ability of Bitoran Specific to the Serine Protease Activity of Human Blood Coagulation Factor Xa

The purified sample of lyophilized bitoran protein obtained from the crude venom of Bitis arietans arietans purchased from LATOXAN is a mixture of the bitoran D protein and bitoran V protein. The mixture was used for evaluating inhibitory potency against the serine protease activity of human blood coagulation factor Xa.

In each well in a 96-well microtiter plate is placed 90 μL of an enzyme solution prepared by adding and dissolving 0.3 μM human factor Xa and a given concentration of a test substance in a Tris-HCl buffered saline (50 mM Tris-HCl, 100 mM NaCl, pH8.0), and the solution is incubated at 37° C. for 15 min. Next, to each well is added 10 μL of a synthetic substrate solution prepared by dissolving a synthetic substrate LGR-PNA in the Tris-HCl buffered saline at a concentration of 4 mM. In each well, the synthetic substrate LGR-pNA at the C-terminal side of Arg residue is cleaved by the protease activity of factor Xa to give p-nitroaniline, whose amount is monitored by observing light absorption at 405 nm and 492 nm using a plate reader. A rate V of formation of p-nitroaniline is calculated, and with reference to a formation rate V₀ in a reference control without the test substance, a reduced protease activity (V/V₀) is calculated.

A trypsin inhibitor substance from plant; SBTI (Soybean Trypsin Inhibitor; MW=20.1 kDa) was used as a positive control, and comparison of the lyophilized “bitoran” protein sample (MW=11 kDa) from the crude venom of Bitis arietans arietans therewith was made to evaluate inhibitory potency of bitoran. FIG. 4 shows reduced protease activities (V/V₀) when adding a test substance at various concentrations. Based on the results shown in FIG. 4, 50% inhibition concentrations (IC₅₀) were determined. As a result, IC₅₀ of SBTI is 3×10⁻⁷ M, while IC₅₀ of the bitoran protein (a mixture of the bitoran D protein and bitoran V protein) is 2×10⁻⁸ M.

Furthermore, inhibitory potency was evaluated in very similar manner using the sample of lyophilized bitoran protein purified from the crude venom of Bitis arietans obtained from the Japan Snake Institute, and IC₅₀ of the bitoran protein (bitoran V protein) was 2×10⁻⁸ M.

Cloning of a Gene Encoding the Bitoran

The bitoran proteins isolated from the venom produced by Bitis arietans are considered to belong to the same family as Kunitz-type proteins having various trypsin inhibitory activities which have been already reported. Specifically, the protein is predicted to be translated as a precursor protein having a leader sequence or signal sequence at its N-terminus, and when being extracellularly secreted, subjected to post-translation processing, resulting in truncation of the N-terminal region. Then, in association with formation of intramolecular Cys-Cys bonding (S—S bond), the protein is predicted to fold in the shape of mature protein having its appropriate three-dimensional configuration.

Cloning was attempted for a gene encoding the precursor protein of the bitoran protein.

A frozen poison gland of Bitis arietans was purchased from SAV Inc., South Africa. Total RNA was prepared by extracting the poison gland of Bitis arietans, cDNA of polyA-RNA was prepared by reverse transcription, and cloning was attempted for a gene encoding the precursor protein of the bitoran protein.

(1) Preparation of Total RNA

Poison glands of Bitis arietans (two glands per animal) purchased from SAV Inc. were crushed while being frozen in liquid nitrogen. From the crushed gland material, RNA was extracted using ISOGEN to prepare total RNA.

To the crushed poison gland tissue was added 15 mL of ISOGEN. Using a syringe with a needle, the mixture is repeatedly passed through the syringe by pressure feeding, to homogenize the crushed tissue cell material. A freezing-melting process where the material was frozen in liquid nitrogen and then melted at room temperature was repeated twice, to finish crushing of the residual tissue cells contained in the dispersion of the crushed tissue cell material. The homogeneous crushed cell dispersion thus obtained was transferred into a 50 mL Falcon tube, and after adding 3 mL of chloroform, thoroughly shaken for 15 sec. Then, the mixture is allowed to stand at room temperature for 2 to 3 min to separate phases. Then, it is centrifuged (12,000 rpm) at 4° C. for 10 min. Then, about 20 mL of the supernatant (aqueous phase) is collected into another 50 mL Falcon tube.

To the collected aqueous phase is added 7.5 mL of isopropanol, and the mixture is thoroughly blended. The mixture is maintained at room temperature for 5 to 10 min, while alcohol precipitation proceeds. Then, the mixture is centrifuged (12,000 rpm) at 4° C. for 10 min. After removing the supernatant, a pellet (alcohol precipitation fraction) is collected.

To the pellet (alcohol precipitation fraction) is added 2 mL of ethanol, and the mixture is dispersed and blended by vortex. The dispersion/mixture is centrifuged (12,000 rpm) at 4° C. for 5 min. After removing the supernatant, a pellet (alcohol precipitation fraction) is collected.

After washing, the RNA precipitation pellet is dried by an aspirator for evaporating the residual solvent. To the dried RNA precipitation pellet is added 40 to 100 μL of DEPC-treated pure water being free from RNase, and the mixture is allowed to stand for 10 to 20 min for re-dissolution, to prepare a sample solution of total RNA.

An RNA concentration in the prepared total RNA sample solution is calculated from an absorbance at a wavelength of 260 nm.

The total RNA sample containing 10 μg of the RNA is added to a mixture of 2 μL of 5×MOPS buffer, 3.5 μL of formaldehyde and 10 μL of formamide, and the mixture is homogeneously mixed. Then, to the mixture is added DEPC-treated pure water to 20 μL of the whole liquid volume. The solution containing the total RNA is heated at 65° C. for 15 min for thermal denaturation to convert it into a single-stranded RNA without a double-stranded structure. Subsequently, to the thermally denaturated total RNA solution is added 2 μL of a dye solution for agarose electrophoresis. The mixture is spotted on an agarose gel modified by 6.7% formaldehyde and electrophoresed in 1×MOPS buffer.

After the electrophoresis, the mixture is stained with ethidium bromide for measuring band densities of 28S rRNA and 18S rRNA as indicators by a density reader. The measured values are used to determine quality of the total RNA (a content ratio of rRNA/mRNA).

(2) Preparation of a First Strand cDNA

An mRNA contained in the prepared total RNA is used to prepare a first strand cDNA complementary to its nucleotide sequence.

In the process, RT-PCR is applied using polyA-RNA as a template to prepare cDNA thereof. The RT-PCR reaction may be conducted using a commercially available kit such as Ready to Go RT-PCR beads (Amersham Pharmacia Biotech), High Fidelity RNA PCR kit (Takara Shuzo Co., Ltd.), SMART RACE cDNA amplification kit (Clontech Laboratories, Inc.) and 5′-Full RACE Core set (Takara Shuzo Co., Ltd.) in accordance with an attached standard protocol in each kit. Specifically, standard reaction conditions fit to a reverse transcription reaction are as follows; at 42° C. for 40 min, using 2 μg of the total RNA for Ready to Go RT-PCR beads, at 60° C. for 30 min, using 2 μg of the total RNA for High Fidelity RNA PCR kit, at 42° C. for 90 min, using 2 μg of the total RNA for SMART RACE cDNA amplification kit, and at 50° C. for 60 min, using 6 μg of the total RNA for 5′-Full RACE Core set, respectively.

Here, RT-PCR was applied using polyA-RNA as a template, and when preparing cDNA therefrom, GeneAmp PCR system 9700 (PerkinElmer Inc.) is used to control a reaction solution temperature and a reaction period as described above.

In the reverse transcription reaction, using polyA-RNA as a template, a primer provided in each kit is hybridized to a polyA sequence region at its 3′end, and then a reverse transcriptase is used to elongate a DNA chain having a complementary nucleotide sequence.

(3) Amplification of cDNA Encoding the Precursor Protein for the Bitoran Protein by PCR Reaction

The amino-acid sequence indicates that the bitoran protein from Bitis arietans venom belongs to a family of Kunitz-type protease inhibitor proteins. Meanwhile, there have been reported several Kunitz-type protease inhibitor proteins from a venom and also deduced amino-acid sequences of precursor proteins on basis of the nucleotide sequences of their coding genes.

FIG. 6 show the results of alignment of the amino-acid sequences among these Kunitz-type mature proteins and the precursor proteins. The alignment indicates that the amino-acid sequences of precursor proteins have a partial amino-acid sequence showing high homology in the N-terminal region, which is considered to be a portion of a leader sequence or a signal sequence. Specifically, in any of the precursor proteins, 15 amino-acid residues from its N-terminus is the sequence “MSSGGLLLLLGLLTL” (SEQ ID NO:17).

We have supposed that in the bitoran protein from the venom of Bitis arietans, a precursor protein thereof also has an N-terminal region corresponding to its leader sequence or signal sequence, which is equivalent to said sequence. Based on the supposition, we have designed, as an upstream primer (sense primer), a primer having the following nucleotide sequence:

(SEQ ID NO:1)

5′-ATG TCT TCT GGA GGT CTT CTT CTC CTG CTG-3′
Met Ser Ser Gly Gly Leu Leu Leu Leu Leu

which corresponds to the nucleotide sequence encoding the 1 to 10 amino-acid residue portion contained in the sequence “MSSGGLLLLLGLLTL” (SEQ ID NO:17). We have requested Hitachi Instruments Service Co., Ltd. to synthesize and purify the sense primer.

By using the commercially available kit: Ready to Go RT-PCR beads, the first strand cDNA prepared from the polyA-RNA contained in the total RNA was used as a template to amplify the cDNA containing the nucleotide sequence of said sense primer in accordance with the procedure below. An upstream primer used therefor was the aforementioned sense primer, while a downstream primer was just the primer used for the reverse transcription reaction. Then, 1.25 μL of the first strand cDNA solution was used to prepare 25 μL in total of a PCR reaction solution containing 10 mM of dNTPs, 10 μM of the upstream primer, 10 μM of the downstream primer, 1 unit/μL of Ex-taq ploymerase and 10×PCR buffer (at final concentration: 1×), and PCR reaction was effected by using the following heat cycle. The heat cycle was as follows; denature: 96° C. for 30 sec, annealing: 53° C. for 30 sec, extension: 72° C. for 60 sec; and 35 cycles in total. Control of the reaction solution temperature and reaction period was carried out using GeneAmp PCR system 9700 (PerkinElmer Inc.).

After the PCR reaction, the reaction solution was collected and electrophoresed on a 1.5% Agarose gel, to determine the presence of a PCR amplification product and its molecular weight. The double-stranded DNA in the PCR amplification product was stained by a 0.5 μg/mL solution of ethidium bromide, and band position thereof was identified using the fluorescence straining under UV irradiation.

(4) Collection of the PCR Amplification Product on the Agarose Gel

While observing the double-stranded DNA band of the identified PCR amplification product under UV irradiation from a UV transiluminator (wavelength λ=302 nm), the gel portion of the band area was cut off by a cutting knife. From the gel piece cut off, the double-stranded DNA was extracted and purified using a commercially available extraction kit: QIAquick Gel Extraction kit (QIAGEN Inc.) in accordance with an attached standard protocol in the kit.

(5) Insertion of the cDNA Fragment into a Cloning Vector

Using a commercially available cloning kit: PGEM-T Easy Vector System I (Promega Corporation), the purified double-stranded DNA is inserted into a cloning site in the cloning vector: PGEM T-easy vector. In accordance with the an attached standard protocol in the cloning kit, a ligation reaction of the vector fragment with the cDNA fragment was conducted at 16° C. for 4 hours or longer, using 10 μL of the total amount of the reaction solution.

(6) Preparation of a Transformant Retaining the Cloning Vector with the Inserted cDNA Fragment

After constructing the plasmid vector with the inserted cDNA fragment, a transformant with the introduced plasmid vector was prepared as follows.

6-1: Preparation of Competent Cells

A host E. coli XL-1 Blue strain is allowed to form a colony on a plate medium containing an antibiotic for selection by drug resistance retained by the strain. An inoculum collected from the colony is inoculated to an SOB medium free from the antibiotic, and cultured under shaking at 17.5° C. The culturing is continued for about 30 to 40 hours until a turbidity of the culture medium determined at a wavelength of 600 nm: A600 reaches A600=0.4 to 0.8. Then, the culture is allowed to cool for 10 min, centrifuged at 4° C. for 12 min (2,300 rpm), and then subjected to harvesting the bacteria cells.

The harvested bacteria are suspended in 17 mL of an ice-cooled transformation buffer (TB), and the suspension is allowed to stand for additional 10 min. Again, the bacterium suspension is centrifuged at 4° C. for 12 min (2,300 rpm) and subjected to harvesting the cells. The harvested bacteria are re-suspended in 4.5 mL of an ice-cooled TB. To the suspension is further added 330 μL of DMSO (final concentration: 7%). The mixture is gently mixed and then allowed to cool on ice for 10 min.

Then, 120 to 150 aliquots of the competent-cell suspension thus prepared are dispensed into 1.5 mL tubes, and then frozen in liquid nitrogen. The frozen competent-cell suspension is stored in a freezer at −80° C.

6-2: Transformation

After the ligation reaction, the plasmid vector collected from the reaction solution is introduced into the competent cell to produce a transformed strain, as described below.

The frozen competent-cell suspension is quickly thawed. Then, the competent-cell suspension is added to the collected plasmid vector in 60 to 100 μL/100 pg, and the mixture is allowed to stand on ice for 30 min. Subsequently, the mixture is subjected to heat shock in a warm bath at 42° C. for 40 sec, and again allowed to stand on ice for 2 min.

The suspension subjected to the heat-shock treatment is diluted with 50 to 100 μL of an LB medium. The diluted cell suspension is applied on an LB plate containing ampicillin which is a target antibiotic of the ampicillin resistance gene contained in the plasmid vector as a selection marker. After culturing at 37° C. overnight, a colony is formed on the LB plate.

(7) Preparation of a Plasmid

The transformed strain retaining PGEM T-easy vector with the inserted cDNA fragment or retaining PGEM T-easy vector forms a colony on the LB plate containing ampicillin. A transformed strain is harvested from the colony and inoculated in the LB medium (10 mL) containing ampicillin (50 to 100 μg/mL). After cultured by shaking at 37° C. overnight, the culture is centrifuged at 4° C. for 15 min (3,000×g) and subjected to harvesting bacteria cells. The harvested cells of the transformed strain is crushed, and the plasmid vector contained therein is separated and purified using a commercially available plasmid purification kit: QIAprep Spin Miniprep Kit (QIAGEN Inc.).

For a purified plasmid prepared from a transformed strain in each colony, whether the plasmid contains a desired cDNA fragment or not is determined by measuring its molecular weight.

(8) Sequencing of the Nucleotide Sequence of the Inserted cDNA

Using the purified plasmid with insertion of the desired cDNA fragment, the nucleotide sequence of the DNA fragment inserted in the PGEM T-easy vector is analyzed as described below.

The nucleotide sequence of the inserted cDNA fragment contains in its 3′end the polyA sequence and the nucleotide sequence derived from the primer contained in the RT-PCR kit which was used in the reverse transcription reaction. The first strand cDNA prepared for the polyA-RNA contained in the total RNA using the commercially available kit: Ready to Go RT-PCR beads contains the nucleotide sequence derived from the M13 reverse primer used in the kit. Using a primer having the nucleotide sequence derived from the M13 reverse primer as a sequencing primer, the nucleotide sequence up to the 5′ end portion that is derived from the upstream primer (sense primer): 5′-ATG TCT TCT GGA GGT CTT CTT CTC CTC CTG CTG-3′ (SEQ ID NO:6) is analyzed.

Specifically, a primer having the nucleotide sequence derived from the M13 reverse primer is used as a sequencing primer, to effect a nucleotide elongation reaction for sequencing, employing the inserted DNA fragment region as a template. At the 3′ end of the elongated chain obtained by the nucleic-acid elongation reaction for sequencing, a fluorescence-labeled nucleotide is introduced. The nucleotide elongation reaction for sequencing is effected using a commercially available kit: Thermo Sequenase kit with 7-deaza-dGTP (Amersham-Pharmacia Inc.), and the sequencing is effected in accordance with a protocol for a sequencer from Shimadzu Corporation.

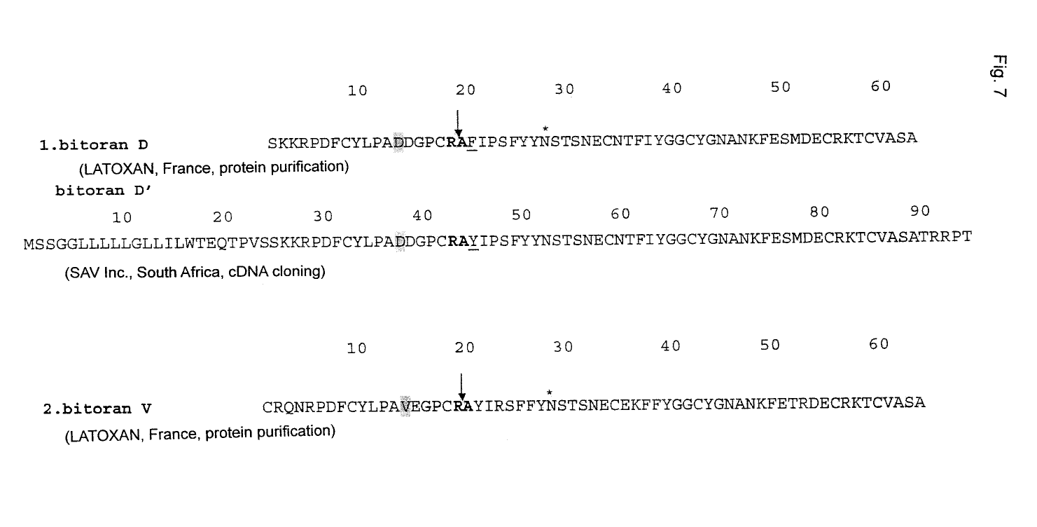

In the cDNA prepared from the mRNA extracted from the poison gland of Bitis arietans purchased from SAV Inc., there was obtained a DNA fragment having the nucleotide sequence shown in FIG. 7 as a PCR amplification product when using the upstream primer (sense primer): 5′-ATG TCT TCT GGA GGT CTT CTT CTC CTC CTG CTG-3′ (SEQ ID NO:6). The nucleotide sequence encodes 89 amino acids, whose (deduced) amino-acid sequence has high homology to the amino-acid sequences of the Kunitz-type mature proteins from other venoms and precursor proteins thereof as shown in FIG. 5.

When aligning the amino-acid sequences of bitoran D protein and bitoran V protein obtained from the venom of Bitis arietans arietans purchased from LATOXAN together with the amino-acid sequence of the bitoran precursor protein encoded by the mRNA collected from the poison gland of Bitis arietans purchased from SAV Inc., the amino-acid sequence of the bitoran precursor protein substantially corresponds to the amino-acid sequence of bitoran D protein as shown in FIG. 7, although Cys⁴⁰-Arg⁴¹-Ala⁴²-Tyr⁴³-Ile⁴⁴ (SEQ ID NO:12) in the bitoran precursor protein (bitoran D′) is corresponding to the region of Cys¹⁷-Arg¹⁸-Ala¹⁹-Phe²⁰-Ile²¹ (SEQ ID NO:11) in the bitoran D protein. It is supposed that the bitoran precursor protein (bitoran D′) is a precursor protein for the bitoran D type protein from Bitis arietans purchased from SAV Inc.

### INDUSTRIAL APPLICABILITY

Bitoran proteins from Bitis arietans venom according to the present invention, particularly the bitoran D protein and bitoran V protein have high inhibitory ability specific to the serine protease activity of human blood coagulation factor Xa. The proteins can be, therefore, used as an inhibitor against human blood coagulation factor Xa, which is available for preventing the process of thrombin formation from pro-thrombin (a thrombin precursor).

1. Bitoran D protein being a Kunitz-type trypsin-inhibitor-like protein showing high inhibitory potency against serine protease activity of human blood coagulation factor Xa, which protein is contained in Bitis arietans venom,
wherein the protein is a Kunitz-type protein having the amino-acid sequence represented by SEQ ID NO: 1.

2. Bitoran V protein being a Kunitz-type trypsin-inhibitor-like protein showing high inhibitory potency against serine protease activity of human blood coagulation factor Xa, which protein is contained in Bitis arietans venom,
wherein the protein is a Kunitz-type protein having the amino-acid sequence represented by SEQ ID NO:2.

3. A variant of bitoran D protein being a Kunitz-type trypsin-inhibitor-like protein showing high inhibitory potency against serine protease activity of human blood coagulation factor Xa, which protein is contained in Bitis arietans venom,
wherein the variant protein is a Kunitz-type protein having the amino-acid sequence represented by SEQ ID NO:3.

4. A modified protein derived from bitoran D being a Kunitz-type trypsin-inhibitor-like protein showing high inhibitory potency against serine protease activity of human blood coagulation factor Xa, which protein is contained in Bitis arietans venom,
wherein the modified protein is a Kunitz-type protein having the amino-acid sequence represented by SEQ ID NO:7.

5. A modified protein derived from bitoran V being a Kunitz-type trypsin-inhibitor-like protein showing high inhibitory potency against serine protease activity of human blood coagulation factor Xa, which protein is contained in Bitis arietans venom,
wherein the modified protein is a Kunitz-type protein having the amino-acid sequence represented by SEQ ID NO:8.

6. Use of the bitoran protein or modified protein thereof as claimed in any of claims 1 to 5 for preparing an inhibitory composition against human blood coagulation factor Xa, wherein the bitoran protein or modified protein thereof is used as an active ingredient having inhibitory activity to serine protease activity of human blood coagulation factor Xa.
